(19) United States
(12) Patent Application Publication
(10) Pub. No.: US 2022/0175411 A1
(43) Pub. Date: Jun. 9, 2022

(54) STENT RETRIEVER WITH EMBOLIC PREVENTION DEVICE

(71) Applicant: KOSWIRE INC., Flowery Branch, GA (US)

(72) Inventors: Jin Park, Morris Plains, NJ (US); Daniel Olsen, Califon, NJ (US); Richard Webster Crowley, Hinsdale, IL (US); Cory Zegel, Hoboken, NJ (US); Mitch Berger, San Francisco, CA (US)

(21) Appl. No.: 17/115,111

(22) Filed: Dec. 8, 2020

(51) Int. Cl. A61B 17/3207; A61F 2/95

(52) U.S. Cl. CPC A61B 17/3207; A61B 2017/320716; A61F 2002/9528; A61F 2/95

(57) ABSTRACT

The present invention pertains to a thrombectomy device comprising a cylindrical proximal portion, a dome-shaped distal portion, a transition portion, and a coil. The cylindrical proximal portion forms a stent frame having a first lattice network of a first plurality of interconnecting segments. The first plurality of interconnecting segments is configured to exert a first radial force against an inner wall of a blood vessel. The dome-shaped distal portion forms a protection cage having a second lattice network of a second plurality of interconnecting segments. The second plurality of interconnecting segments is configured to exert a second radial force against the inner wall of the blood vessel. The transition portion is arranged between the stent frame and the protection cage. The coil is formed at a distal end of the protection cage.

### TECHNICAL FIELD

The present invention is generally related to the field of catheter-based removal of unwanted matter from cerebral vascular structures and more particularly to stent retrievers having an embolism prevention device that prevents fragments of the unwanted matter from lodging in the cerebral vascular structures.

### BACKGROUND

Stent retriever technology has historically been used to remove thrombus (i.e., blood clot) from the neurovasculature (i.e., blood vessels in the brain) in a procedure known as neurothrombectomy for the treatment of strokes. The thrombus causes occlusion of the blood vessel, restricting blood flow and significantly reducing oxygen and nutrient delivery to surrounding tissue. As the thrombus persists, tissue distal to the thrombus experiences necrosis (i.e., cellular death), which can ultimately lead to brain damage or death, amongst other negative conditions. Stent retriever technology is intended to capture the thrombus and remove it from the effected blood vessel, thereby returning normal flow to the vessel and preventing further necrosis of the tissue.

Current technology involves expanding a metal device into the thrombus to secure itself, and then removing the device and the thrombus in tandem. However, due to the mechanical nature in which the device embeds into the thrombus, it is possible that fragmentation of the thrombus may occur, creating an embolus which can travel further downstream known as a secondary emboli. The embolus may attach itself to another vessel, causing embolization of another artery and impacting patient safety. Current technology for mitigating the risk of emboli formation involves simultaneous aspiration of the blood vessel while deploying the stent retriever. Simultaneous aspiration increases (1) the difficulty the procedure and (2) may take additional time to restore blood flow without the guarantee of capturing all thrombus fragments.

In regions of the vasculature with larger diameter vessels (i.e., below the waist), a distal embolic protection device may be used to mitigate the risk of secondary emboli. Due to the size constraints imposed by working in the neurovasculature, current iterations of the distal embolic protection device cannot be used in neurothrombectomy procedures. Current iterations of the devices are too large to fit in the smaller blood vessels in the brain and the introduction of secondary devices in small vessels increases both difficulty of the procedure and risk to the patient as it will take longer to restore blood flow as well increasing the potential to damage the blood vessel walls.

A thrombectomy device has been theorized in prior art 1, reference patent number U.S. Pat. No. 8,632,584. The prior art 1 theorizes a longitudinally open tube with interconnected strings or filaments forming a mesh structure designed to capture thrombus in small-lumen intra-cranial vessels. The device lacks any form of distal protection, forcing the clinician to use secondary products and more complicated procedural steps to prevent the loss of secondary emboli during the procedure. Additionally, the device is radially rigid, such that if it was impeded form opening in any one location, the entire structure would be impeded from opening, resulting in overall reduced clinical efficacy.

A thrombectomy device used in conjunction with a distal protection device has been theorized in prior art 2, reference patent number U.S. Pat. No. 9,445,829. The prior art 2 theorizes a distal net connected to the thrombectomy device through the usage of multi-layered members (inner and outer members). Having multiple stacked members (inner and outer members) decreases the space available for blood flow, impeding the patients ability to deliver nutrients downstream. Having multiple stacked members also increases the outer diameter of the device, increasing device size and surgical difficulty while limiting access to smaller vessels. The stacked members may also prohibit the device from being fully retracted into the microcatheter, forcing the physician to retract the device and the microcatheter in tandem. This may lead to additional secondary emboli, vessel damage from the device, loss of the thrombus, and potential additional procedural time if access to the vessel is still required.

A combination of a thrombectomy device with distal protection has been theorized in prior art 3, reference patent number U.S. Pat. No. 9,456,834. The prior art 3 theorizes a distal mesh or small pore structure used to capture secondary emboli. The method of manufacturing such a device involves the combination of separate devices, therefore increasing risk of mid-procedure malfunction such as breakage of the distal mesh from the stent frame. Additionally, multi-part construction theoretically increases device size, increasing surgical difficulty and limiting access to smaller vessels.

Therefore, there is a need for a thrombectomy device that both reduces the risk of emboli escaping the thrombus site by using an embolus protection mechanism and is capable of being used in smaller vessels without risk of breakage or malfunction.

### SUMMARY

At least the above-discussed need is addressed and technical solutions are achieved in the art by various embodiments of the present invention. Some embodiments of the present invention pertain to a thrombectomy device comprising a cylindrical proximal portion forming a stent frame having a first lattice network of a first plurality of interconnecting segments, the first plurality of interconnecting segments being configured to exert a first radial force against an inner wall of a blood vessel; a dome-shaped distal portion forming a protection cage having a second lattice network of a second plurality of interconnecting segments, the second plurality of interconnecting segments being configured to exert a second radial force against the inner wall of the blood vessel; a transition portion arranged between the stent frame and the protection cage; and a coil formed at a distal end of the protection cage.

In some embodiments of the invention, the first plurality of interconnecting segments is arranged to include openings when the stent frame of the thrombectomy device is deployed in an open position and the second plurality of interconnecting segments is arranged to include openings when the protection cage of the thrombectomy device is deployed in an open position.

In some embodiments of the invention, the stent frame can be deployed in the open position independently of the protection cage being deployed in the open position.

In some embodiments of the invention, the protection cage is deployed in the open position before the stent frame is deployed in the open position.

In some embodiments of the invention, the openings in the stent frame have a larger cross-section than the openings in the protection cage.

In some embodiments of the invention, a size of the openings in the stent frame and the openings in the protection cage decreases down a gradient from a proximal end of the stent frame towards an apex of the protection cage.

In some embodiments of the invention, the thrombectomy device further includes a microcatheter designed to deliver and retrieve the thrombectomy device from the blood vessel in a closed position.

In some embodiments of the invention, the first plurality of interconnecting segments is arranged to be fully connected without any openings when the stent frame of the thrombectomy device is deployed in a closed position, and the second plurality of interconnecting segments is arranged to be fully connected without any openings when the protection cage of the thrombectomy device is deployed in a closed position.

In some embodiments of the invention, the stent frame, the transition portion, the protection cage, and the coil are formed of a single piece construction.

In some embodiments of the invention, outer diameter of the stent frame is smaller than an outer diameter of the protection cage.

In some embodiments of the invention, a ratio of the length of the transition portion to a length of the stent frame is between 1:20 and 1:8.

In some embodiments of the invention, the thrombectomy device further includes a plurality of radiopaque markers positioned on the stent frame to permit a position of the stent frame to be viewed in vivo.

In some embodiments of the invention, the coil is radiopaque to permit a position of the coil to be viewed in vivo.

In some embodiments of the invention, the openings formed in the second lattice network are sized such that the protection cage captures emboli without preventing blood flow past the protection cage.

In some embodiments of the invention, the stent frame is configured to capture a thrombus in the blood vessel, and the protection cage is configured to capture at least on embolus formed by a breaking of the thrombus in the blood vessel.

These and other embodiments of the invention are discussed in detail below.

### BRIEF DESCRIPTION OF THE DRAWINGS

It is to be understood that the attached drawings are for purposes of illustrating aspects of various embodiments and may include elements that are not to scale. It is noted that like reference characters in different figures refer to the same objects.

FIG. 1 depicts a side view of a thrombectomy device, according to an embodiment of the invention.

FIG. 2 depicts a side view of another thrombectomy device, according to an embodiment of the invention.

FIG. 3 depicts a side view of another thrombectomy device, according to an embodiment of the invention.

FIG. 4 depicts an end view of a distal end of a thrombectomy device, according to an embodiment of the invention.

FIG. 5 depicts an end view of a distal end of another thrombectomy device, according to an embodiment of the invention.

FIG. 6 depicts a distal tip coil on the distal end of a thrombectomy device, according to an embodiment of the invention.

FIG. 7 depicts a cross section of a distal tip coil on the distal end of a thrombectomy device, according to an embodiment of the invention.

FIG. 8 depicts a representative image of a blood vessel obstructed by a thrombus, preventing blood flow to the distal end of the blood vessel, according to an embodiment of the invention.

FIG. 9 depicts the obstructed blood vessel from FIG. 8 during the first step of a treatment process in which a microcatheter is inserted into the proximal end of the thrombus until it exits beyond the distal end of the thrombus according to an embodiment of the invention.

FIG. 10 depicts the obstructed blood vessel from FIG. 8 during the second step of the treatment process in which the thrombectomy device is advanced through the microcatheter and positioned across the thrombus and surrounding areas, according to an embodiment of the invention.

FIG. 11 depicts the obstructed blood vessel from FIG. 8 during the third step of the treatment process in which secondary emboli are trapped in the distal portion of the thrombectomy device, according to an embodiment of the invention.

FIG. 12 depicts a representative image of the blood vessel from FIG. 8 following removal of the thrombectomy device and the thrombus after completion of the treatment process, according to an embodiment of the invention.

### DETAILED DESCRIPTION

In the descriptions herein, certain specific details are set forth in order to provide a thorough understanding of various embodiments of the invention. However, one skilled in the art will understand that the invention may be practiced at a more general level without one or more of these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily all referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects.

In the following description, the phrase “at least” is or may be used herein at times merely to emphasize the possibility that other elements may exist beside those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘including at least A’ includes A as well as the possibility of one or more other additional elements besides A. In the same manner, the phrase, ‘including A’ includes A, as well as the possibility of one or more other additional elements besides A. However, the phrase, ‘including only A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

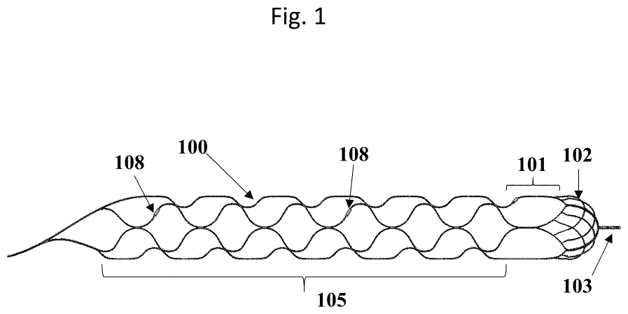

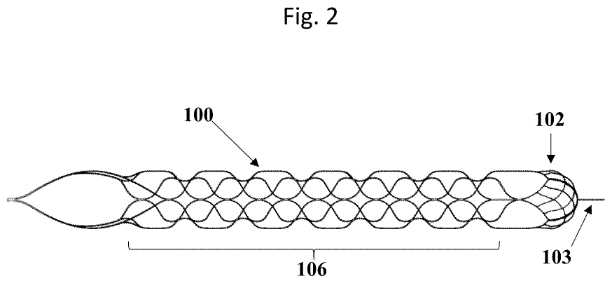

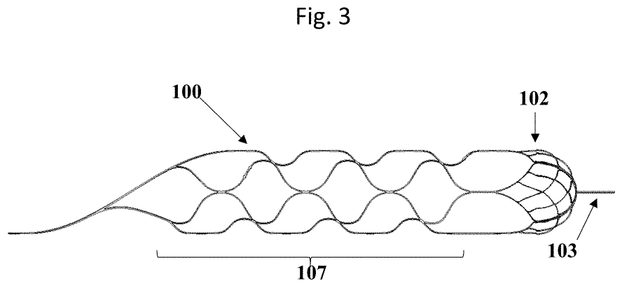

FIG. 1 shows a thrombectomy device according to some embodiments of the present invention. The thrombectomy device includes a stent retriever frame 100 having a distal protection cage 102 which terminates into a distal tip 103. In some embodiments, the distal tip 103 may be radiopaque. In some embodiments, the thrombectomy device is formed of a single piece construction, with both the stent retriever frame 100 and distal protection cage 102 formed continuously without the need for connectors or weld joints. In some embodiments, the stent retriever frame 100 is composed of a lattice network of either non-rigid or rigid interconnected metallic segments designed to open to the vessel wall with a radial force significant enough to penetrate a blood clot but not damage the surrounding blood vessel. In some embodiments of the invention, the stent retriever frame 100 is open such that the metallic segments are not fully connected in the radial direction to facilitate integration into the thrombus. In some embodiments of the invention, the stent retriever frame 100 is closed such that the metallic segments are fully connected in the radial direction to increase device stability. In some embodiments of the invention, as shown in FIGS. 1-3, the stent retriever frame 100 may be manufactured to various lengths 105, 106, 107 to accommodate different anatomical needs. In some embodiments of the invention, the lattice network of the stent retriever frame 100 may be manufactured to larger or smaller diameters to accommodate different anatomical needs.

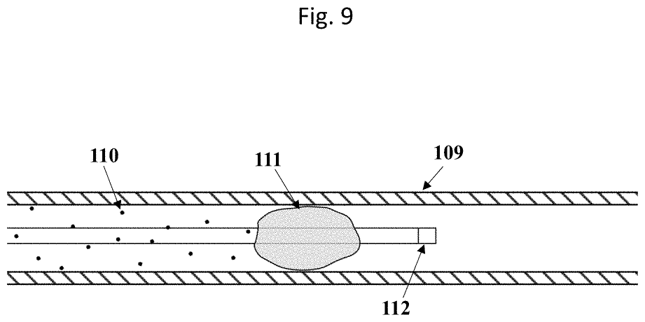

In some embodiments of the invention, the distal portion of the thrombectomy device acts as a distal protection cage 102 designed to entrap secondary emboli 113 generated during the execution of a clinical procedure while still allowing for blood flow 110. The details of the clinical procedure for use of the thrombectomy device are shown in FIGS. 9-13 and discussed later in the specification. In some embodiments of the invention, the distal protection cage 102 is formed as a continuation of the lattice network of metallic segments from the stent retriever frame 100. In some embodiments of the invention, a distal end of the distal protection cage 102 terminates to a singular coil 103.

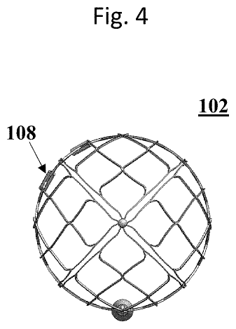

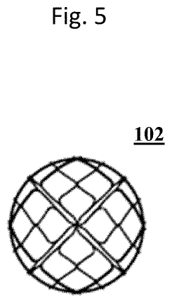

As shown in FIGS. 4 and 5, in some embodiments of the invention, the lattice network of the distal protection cage 102 is rigid in that the lattice network is fully interconnected and funnels to a singular point. In some embodiments of the invention, the lattice network of the distal protection cage 102 has a window (opening) size (distance between metallic segments) smaller than the window (opening) size of the lattice network in a proximal region of the stent retriever frame 100 to ensure secondary emboli 113 will be captured in vivo. The window size decreases down a gradient from a proximal end of the stent retriever frame 100 towards the apex of the distal protection cage 102.

In some embodiments of the invention, each window (opening) of the lattice network of the thrombectomy device has a cross sectional area between 0.5 mm² and 2 mm². In some embodiments of the invention, the distal protection cage 102 has a dome shape to promote even distribution of any secondary emboli captured. Even distribution of the emboli aids in ease of retraction of the thrombectomy device from the blood vessel post procedure. During retraction of the device post procedure, the distal protection cage 102 may compress down to fit into a microcatheter 112, compressing any secondary emboli that have been captured. With a more even distribution of secondary emboli 113, less compression is required, thereby reducing the force required to fully retract the device. In some embodiments of the invention, the outer diameter of the distal protection cage 102 is equal to or greater than the outer diameter of the proximal frame 100.

In some embodiments of the invention, a region between the stent retriever frame 100 and the distal protection cage 102 is referred to as a transition zone 101. The transition zone 101 provides a sufficient distance between the stent retriever frame 100 and the distal protection cage 102 to allow the stent retriever frame 100 and the distal protection cage 102 to open semi-independently of one another. In some embodiments of the invention, the transition zone 101 permits the distal protection cage 102 to open beyond the stent retriever frame 100 to ensure that the distal protection cage's 102 outer diameter will always be equal to or greater than the diameter of the stent retriever frame 100, increasing the likelihood that all secondary emboli may be captured during the procedure.

In some embodiments of the invention, the ratio of the transition zone 101 length to the stent frame 100 length must be at a minimum 1:20 and at a maximum 1:8 to maintain optimal performance. During clinical use, the thrombectomy device may be placed such that the stent retriever frame 100 is positioned inside of a thrombus, and the transition zone 101 and distal protection cage 102 are positioned distal of the thrombus. Due to resistance generated by the thrombus, the stent retriever frame 100 may expand at a rate less than the transition zone 101 and the distal protection cage 102. Clinically, the distal protection cage 102 will open to its full diameter rapidly to ensure that the entire blood vessel 109 is protected from secondary emboli 113 while the stent retriever frame 100 slowly opens and integrates into the thrombus. The transition zone 101 permits the distal protection cage 102 to open to its maximum diameter without creating high stress regions.

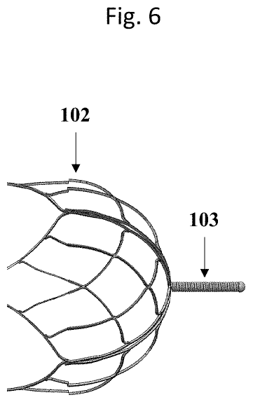

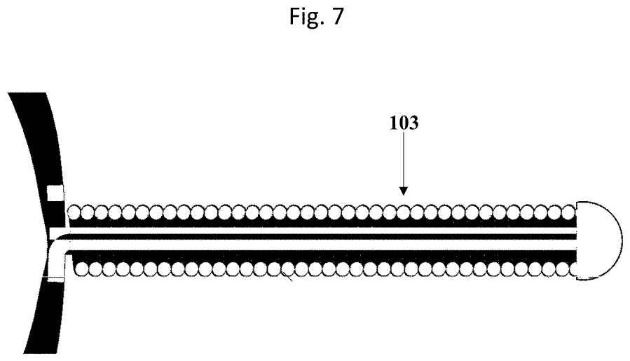

FIGS. 6 and 7 show a distal tip coil 103 formed at a distal tip of the thrombectomy device, according to an embodiment of the invention. The coil 103 surrounds the terminal ends of the distal protection cage 102 and ensures that all lattice members of the distal protection cage 102 come to a singular point. The coil 103 increases stability in the distal protection cage 102 during expansion and retraction of the device. In some embodiments of the invention, the coil 103 is radiopaque, giving the end user increased visibility in vivo. The radiopacity of the coil 103 allows the end user to visualize the most distal end of the device and increases the likelihood that the end user will properly place the thrombectomy device in respect to the thrombus, further improving patient outcomes. In some embodiments of the invention, additional radiopaque markers 108 may be placed throughout the stent retriever frame 100 or the distal protection cage 102 to further aid in visibility and ease of use. The number of radiopaque markers 108 may be dependent upon the length of the thrombectomy device, with more markers being placed on thrombectomy devices with longer overall lengths.

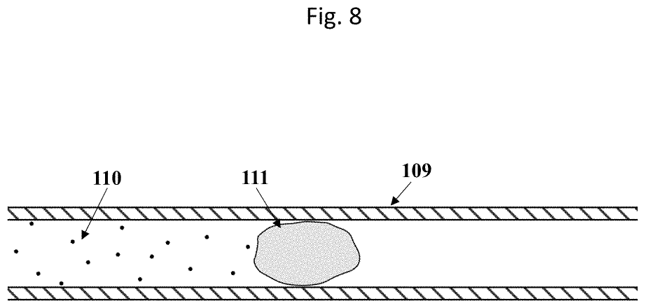

FIGS. 8-12 show cross sectional views of a blood vessel during a treatment process using the thrombectomy device. FIG. 8 depicts a representative image of a blood vessel 109 obstructed by a thrombus 111, preventing blood flow 110 to the distal end of the blood vessel, according to an embodiment of the invention.

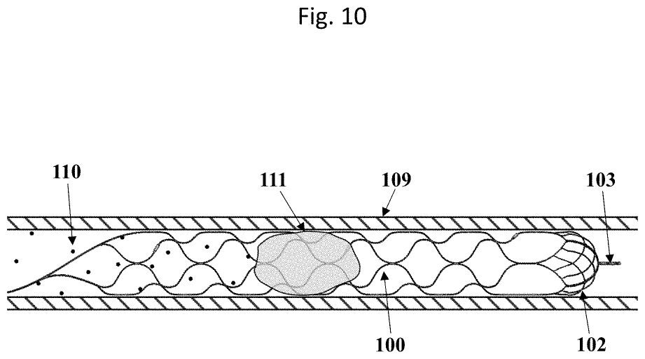

In some embodiments of the invention, the thrombectomy device is designed to treat a thrombus 111 in a blood vessel 109 where the thrombus 111 has impeded blood flow 110 to the distal end of the blood vessel 109. As shown in FIG. 9, in a first step of the treatment process, a microcatheter 112 is inserted into the effected blood vessel 109 distally through the thrombus 111. FIG. 10 shows the blood vessel 109 during a second step of the treatment process. In some embodiments of the invention, the thrombectomy device is advanced through the microcatheter 112 such that the stent frame 100 is both proximal and distal to the thrombus 111 simultaneously, and the distal protection cage 102 is distal to the thrombus 111.

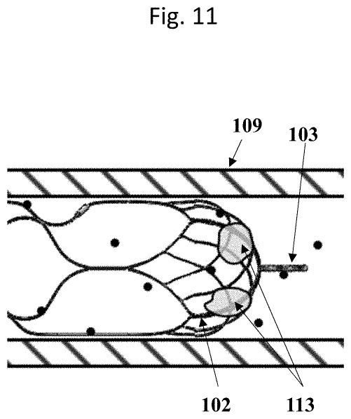

In some embodiments of the invention, the thrombectomy device is designed such that it is self-expanding upon removal of a delivery sheath. The stent retriever frame 100 and the distal protection cage 102 are designed to open up to a full vessel diameter, with different embodiments of the thrombectomy device being capable of expanding to a varying array of diameters to suit patient needs. In some embodiments of the invention, the stent retriever frame 100 exerts sufficient radial force to penetrate and integrate into a thrombus 111 without damaging the vessel wall 109. The thrombectomy device is designed such that if secondary emboli 113 are generated during the procedure, they will be captured and evenly dispersed through the distal protection cage 102 as shown in step 3 of the treatment process depicted in FIG. 11.

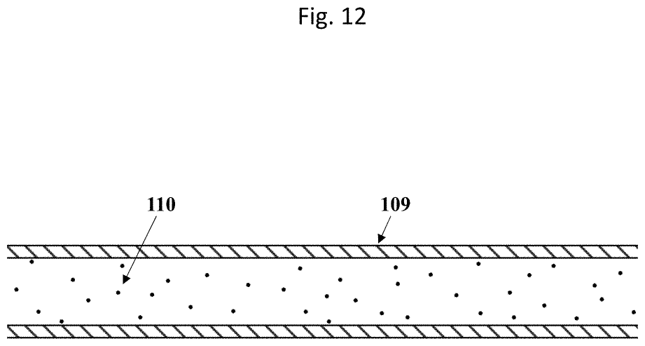

In some embodiments of the invention, after self-expansion and integration into the thrombus 111, the thrombectomy device may be retracted into the microcatheter 112, re-folding into its original compressed configuration with the thrombus 111 and any secondary emboli 113 incorporated into the structure. As shown in FIG. 12, following removal of the device through the microcatheter 112, blood flow 110 is restored to the vessel 109 allowing for the delivery of critical nutrients to areas distal to treatment site. In some embodiments of the invention, the thrombectomy device is attached to a push wire used to advance the thrombectomy device to the target location through a microcatheter 112.

It should be understood that the invention is not limited to the embodiments discussed above, which are provided for purposes of illustration only. Subsets or combinations of various embodiments described above provide further embodiments of the invention.

These and other changes can be made to the invention in light of the above-detailed description and still fall within the scope of the present invention. In general, in the following claims, the terms used should not be construed to limit the invention to the specific embodiments disclosed in the specification. Accordingly, the invention is not limited by the disclosure, but instead its scope is to be determined entirely by the following claims.

1. A thrombectomy device comprising:
a cylindrical proximal portion forming a stent frame having a first lattice network of a first plurality of interconnecting segments, the first plurality of interconnecting segments being configured to exert a first radial force against an inner wall of a blood vessel;
a dome-shaped distal portion forming a protection cage having a second lattice network of a second plurality of interconnecting segments, the second plurality of interconnecting segments being configured to exert a second radial force against the inner wall of the blood vessel;
a transition portion arranged between the stent frame and the protection cage; and
a coil formed at a distal end of the protection cage.

2. The thrombectomy device according to claim 1,
wherein the first plurality of interconnecting segments is arranged to include openings when the stent frame of the thrombectomy device is deployed in an open position, and
wherein the second plurality of interconnecting segments is arranged to include openings when the protection cage of the thrombectomy device is deployed in an open position.

3. The thrombectomy device according to claim 2, wherein the stent frame can be deployed in the open position independently of the protection cage being deployed in the open position.

4. The thrombectomy device according to claim 2, wherein the protection cage is deployed in the open position before the stent frame is deployed in the open position.

5. The thrombectomy device according to claim 2, wherein the openings in the stent frame have a larger cross-section than the openings in the protection cage.

6. The thrombectomy device according to claim 2, wherein a size of the openings in the stent frame and the openings in the protection cage decreases down a gradient from a proximal end of the stent frame towards an apex of the protection cage.

7. The thrombectomy device according to claim 1, further including a microcatheter designed to deliver and retrieve the thrombectomy device from the blood vessel in a closed position.

8. The thrombectomy device according to claim 1,
wherein the first plurality of interconnecting segments is arranged to be fully connected without any openings when the stent frame of the thrombectomy device is deployed in a closed position, and
wherein the second plurality of interconnecting segments is arranged to be fully connected without any openings when the protection cage of the thrombectomy device is deployed in a closed position.

9. The thrombectomy device according to claim 1, wherein the stent frame, the transition portion, the protection cage, and the coil are formed of a single piece construction.

10. The thrombectomy device according to claim 1, wherein an outer diameter of the stent frame is smaller than an outer diameter of the protection cage.

11. The thrombectomy device according to claim 1, wherein a ratio of the length of the transition portion to a length of the stent frame is between 1:20 and 1:8.

12. The thrombectomy device according to claim 1, further including a plurality of radiopaque markers positioned on the stent frame to permit a position of the stent frame to be viewed in vivo.

13. The thrombectomy device according to claim 1, wherein the coil is radiopaque to permit a position of the coil to be viewed in vivo.

14. The thrombectomy device according to claim 1, wherein openings formed in the second lattice network are sized such that the protection cage captures emboli without preventing blood flow past the protection cage.

15. The thrombectomy device according to claim 1,
wherein the stent frame is configured to capture a thrombus in the blood vessel, and
wherein the protection cage is configured to capture at least on embolus formed by a breaking of the thrombus in the blood vessel.
